(19) United States
(12) Patent Application Publication
(10) Pub. No.: US 2025/0051298 A1
(43) Pub. Date: Feb. 13, 2025

(54) CONTINUOUS FLOW CHEMISTRY FOR SYNTHESIS OF CANNABINOIDS

(71) Applicant: COLORADO CHROMATOGRAPHY, LLC, Hudson, CO (US)

(72) Inventors: Giovanni A. RAMIREZ, Hudson, CO (US); Tesfay T. TESFATSION, Hudson, CO (US); Ivan CRUCES, Hudson, CO (US); Adam J. HELLMANN, Hudson, CO (US); Kyle P. RAY, Hudson, CO (US); Westley CRUCES, Hudson, CO (US)

(73) Assignee: COLORADO CHROMATOGRAPHY, LLC, Hudson, CO (US)

(21) Appl. No.: 18/799,833

(22) Filed: Aug. 9, 2024

Related U.S. Application Data
(60) Provisional application No. 63/531,670, filed on Aug. 9, 2023.

(51) Int. Cl. C07D 311/80; B01J 27/20

(52) U.S. Cl. CPC C07D 311/80; B01J 27/20; B01J 2523/824

(57) ABSTRACT

Continuous flow chemistry processes are disclosed herein. The continuous flow processes can be used to produce tetrahydrocannabinol from a cannabidiol starting material. The continuous flow processes can also be used to produce hexahydrocannabinol from a tetrahydrocannabinol starting material. Multiple flow chemistry reactor systems can be combined to produce hexahydrocannabinol from a cannabidiol starting material. The continuous flow processes can be used to produce the desired products at kilogram scales within minutes.

This application claims priority of U.S. Provisional Patent Application No. 63/531,670, filed Aug. 9, 2023, which is hereby incorporated by reference in its entirety.

### BACKGROUND OF THE INVENTION

### I. Field of the Invention

This invention relates to the fields of organic chemistry and process chemistry.

### II. Background

Chemical synthesis in the laboratory has been carried out in standardized glassware, and the use of glassware as the primary batch-type laboratory scale reactor has remained vastly unchanged for over 200 years. By contrast, continuous-flow processes are not common in bench laboratories, and are generally found in larger, industrial-scale systems. Production process aspects such as facile automation, reproducibility, safety and process reliability due to constant reaction parameters are associated with larger, industrial-scale flow process systems. Despite the tremendous progress in the development of new chemical methodologies over the past decades, there is a quest for new enabling technologies that allow for these reactions to be scaled up and to simplify purification or isolation of the desired products.

With respect to cannabinoid synthesis, most cannabinoid production firms currently use batch-style process to produce large quantities of materials under long reaction durations. Flow chemistry has been shown to be useful for the production of cannabinoids, albeit at small scales.

Flow chemistry is beneficial because it closes the gap between small-scale bench chemists and large-scale process engineers by mimicking large-scale production in a laboratory environment. The time from bench to large-scale production of fine chemicals and pharmaceuticals is too long because syntheses developed in the laboratory often cannot be transferred into large-scale production without substantial optimization. Small-scale flow processes provide an opportunity to bridge the gap between small, bench-scale and large-scale production processes. By employing flow chemistry processes for the production of cannabinoids, smaller-scale flow productions can be implemented and more rapidly scaled-up to larger scales. There exists a need in the cannabinoid production industry for flow chemistry methodologies that provide simplified and expedited links to accessing large-scale processes.

### SUMMARY OF THE INVENTION

Tetrahydrocannabinol is a partial agonist at the cannabinoid type 1 receptor and has been approved as a therapeutic treatment for a myriad of conditions. The present inventors have developed novel flow chemistry processes techniques and reactor systems for producing various cannabinoid products, including tetrahydrocannabinol and hexahydrocannabinol. They have developed unique reactor systems and techniques that employ nonionizing ultrasound and/or microwave radiation in novel flow chemistry process techniques for producing these cannabinoids in large quantities with decreased reaction times. The flow chemistry processes disclosed herein enable a high degree of precision in the delivery of reagents/solutions and excellent control over the conditions to which the solutions are exposed. Precise control results in excellent reproducibility and safety thereby making the methods disclosed herein amenable to larger and larger scales. The different processes disclosed herein can also be used in tandem, thereby providing a means to perform multi-step syntheses under flow conditions. The continuous flow processes can be used to produce the desired products at kilogram scales within minutes.

Some aspects of the disclosure are directed to a continuous flow process for producing tetrahydrocannabinol from a cannabidiol starting material. In some embodiments, the process comprises mixing a cannabidiol starting material with a solvent to form a solution, adding an acid to the solution to form a reaction solution, feeding the reaction solution through a continuous flow loop, and passing the reaction solution through an energizing component to produce tetrahydrocannabinol. The acid can be a Lewis acid or a Bronsted acid. In some instances, the acid is hydrochloric acid, sulfuric acid, aluminum trichloride, para-toluenesulfonic acid, camphorsulfonic acid, scandium(III) triflate, benzoic acid, trifluoromethanesulfonic acid, trifluoromethanesulfonic anhydride, bismuth tris (trifluoromethanesulfonate), ytterbium (III) trifluoromethanesulfonate, indium (III) trifluoromethanesulfonate, erbium (III) trifluoromethanesulfonate, cerium (III) trifluoromethanesulfonate, samarium (iii) trifluoromethanesulfonate, or any acid that is known in the art to catalyze cyclization to form a carbocyclic ring or heterocyclic ring. In some embodiments, the tetrahydrocannabinol comprises delta-8 tetrahydrocannabinol, delta-9 tetrahydrocannabinol, or a combination thereof.

In some embodiments, the method comprises passing the reaction through the continuous flow loop for a period of time that is any one of, less than, greater than, or between 1, 2, 3, 4, 5, 6, 7, 8, 9, 10, 11, 12, 13, 14, 15, 16, 17, 18, 10, 20, or more minutes, or any range therein. In some embodiments, the method comprises passing the reaction through the continuous flow loop for a period of time ranging from 2 minutes to 20 minutes. In some embodiments, the energizing component is in-line with the continuous flow loop. In some embodiments, the energizing component is a microwave reactor, a sonicator, a heating bath, or a combination thereof. When the energizing component comprises a microwave reactor, at least a portion of the continuous flow loop is microwave-transparent. When the energizing component comprises a heating bath, the process further comprises heating the reaction solution to a temperature ranging from 25° C. to 100° C. In some embodiments, the reaction solution is heated to a temperature that is any one of, less than, greater than, or between 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C., or any range derivable therein. In some embodiments, at least a portion of a flow path within the energizing component is non-linear. In some embodiments, a flow path within the energizing component is coiled. In some embodiments, the solvent is selected from the group consisting of methanol, ethanol, propanol, isopropanol, butanol, tert-butanol, THF, 2-Me-THF, toluene, and ethyl acetate.

Some aspects of the disclosure are directed to a continuous flow process for producing hexahydrocannabinol from a tetrahydrocannabinol starting material. In some embodiments, the process comprises mixing a tetrahydrocannabinol starting material with a solvent to form a first solution, feeding the first solution into a continuous flow loop, feeding hydrogen gas into the continuous flow loop to form a combined feed comprising the hydrogen gas and the first solution, and, passing the combined feed over a catalyst bed within the continuous flow loop to produce hexahydrocannabinol. In some embodiments, the tetrahydrocannabinol starting material is delta-8 tetrahydrocannabinol, delta-9 tetrahydrocannabinol, or a mixture thereof.

In some embodiments, the catalyst bed is provided on a candle filter, Nutsche filter, ZWAG filter, or FUNDA filter support. In some embodiments, the catalyst bed is provided on a cartridge-type filter that can be based on any of the aforementioned filter types. In some aspects, any combination of the aforementioned filters and/or cartridges can be arranged in tandem and/or parallel loop systems. In some embodiments, the catalyst is selected from the group consisting of Pd/C, Pt/C, Rh/C, Ru/C, Ir/C, Ni/C, Fe/C, Co/C, V/C, Mn/C, Raney nickel, Raney cobalt, Pd/alumina, Pt/alumina, Pt/activated charcoal, Pt₂O (Adam's catalyst), Wilkinson's catalyst ([RhCl(PPh₃)₃]), Crabtree's catalyst ([C₈H₁₂IrP(C₆H₁₁)₃C₅H₅N]PF₆), 9-borabicyclo[3.3.1]nonane, alpine borane, BH₃-DMSO, BH₃-THF, and N-methylimidodiacetic (MIDA) boronates, tetrakis(triphenylphospine)palladium, metal on alumina, metal on activated charcoal including Pd/activated charcoal, metal oxides, metal hydroxides, metal salts, metal halides, and metal acetates. In some embodiments, the solvent is selected from the group consisting of methanol, ethanol, propanol, isopropanol, butanol, tert-butanol, THF, 2-Me-THF, toluene, and ethyl acetate. In some embodiments, the method further comprises heating the catalyst to a temperature ranging from 25° C. to 100° C. In some embodiments, the catalyst is heated to a temperature that is any one of, less than, greater than, or between 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100° C., or any range derivable therein. In some embodiments, no heat is supplied to any component of the continuous flow reactor. In some embodiments, the continuous flow reactor is purged with an inert gas prior to addition of reactants and/or catalyst. The inert gas can be argon, nitrogen, xenon, or any other gas that is not reactive in the continuous flow system. Pressure within any component of the continuous flow reactor can be varied. The pressure within any component of the continuous flow reactor can be any one of, less than, greater than, between, or any range thereof of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 psi, or any range derivable therein.

Some aspects of the present disclosure are directed to a bifurcated continuous flow process for producing hexahydrocannabinol from a cannabidiol starting material. In some embodiments, the process comprises mixing a cannabidiol starting material with a solvent to form a solution, adding an acid to the solution to form a reaction solution, feeding the reaction solution through a first continuous flow loop, passing the reaction solution through an energizing component to produce tetrahydrocannabinol, feeding the reaction solution comprising tetrahydrocannabinol through a second continuous flow loop, feeding hydrogen gas into the second continuous flow loop to form a combined feed comprising the hydrogen gas and the reaction solution comprising tetrahydrocannabinol, and passing the combined feed over a catalyst bed within the second continuous flow loop to produce hexahydrocannabinol.

In some embodiments, the process comprises continuously flowing the reaction solution through the first continuous flow loop until a tetrahydrocannabinol yield of at least 95% has been reached. The process can comprise continuously flowing the reaction solution through the first continuous flow loop until a tetrahydrocannabinol yield that is any one of, less than, greater than, between, or any range thereof of 95%, 96%, 97%, 98%, 99%, or 100%, or any range derivable therein. In some embodiments, the process further comprises continuously flowing the reaction solution through the first continuous flow loop until a hexahydrocannabinol yield of at least 95% has been reached. The process can comprise continuously flowing the reaction solution through the first continuous flow loop until a hexahydrocannabinol yield that is any one of, less than, greater than, between, or any range thereof of 95%, 96%, 97%, 98%, 99%, or 100%, or any range derivable therein. In some embodiments, the process further comprises passing the reaction solution comprising tetrahydrocannabinol through a filter prior to feeding the reaction solution into the second continuous flow loop to remove at least a portion of the acid.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a diagram depicting a six-component flow system. The flow design provides the ability to tailor the flow path and add additional components that lead to the final product,

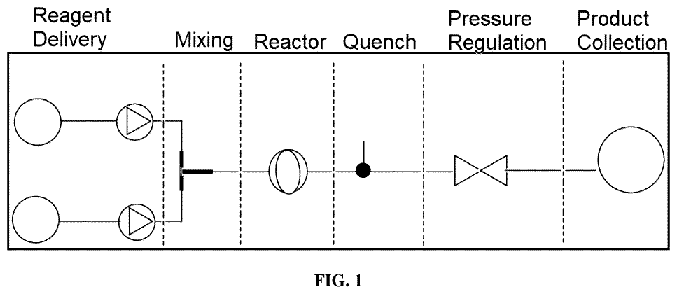

FIGS. 2A-2B—FIG. 2A is a diagram depicting microenvironments of solvated compounds caused by sonication. FIG. 2B is a diagram comparing heating introduction and temperature distribution resulting from conventional heating (top) and microwave heating (bottom).

FIG. 3 is a diagram depicting a microwave-assisted continuous flow reactor that can be used to produce a mixture of Δ-8 tetrahydrocannabinol and Δ-9 tetrahydrocannabinol from cannabidiol.

FIG. 4 is a diagram depicting products that can be obtained from acid-catalyzed cyclization of cannabidiol.

FIG. 5 is a diagram depicting mechanisms for the transformation of cannabidiol into iso-tetrahydrocannabinol (top) and tetrahydrocannabinol (bottom).

FIGS. 6A-6B—FIG. 6A is a diagram depicting microwave effects on the polarizability of polar solvents, as well as local heating or superheating causing increased reaction rates. FIG. 6B is a diagram depicting acoustic waves from a sonicator that cause compression and rarefaction, leading to increased mechanical effects and increased reaction kinetics.

FIGS. 7A-7B. FIG. 7A is a diagram depicting the conversion of CBD to THC over time, as produced by a microwave-assisted continuous flow reactor. FIG. 7B is a diagram depicting the conversion of CBD to THC over time, as produced by a sonicator-assisted continuous flow reactor.

FIG. 8 is a schematic of a microwave oven that has been modified with holes drilled into the top, in order to allow condenser and thermocouple patch thermometer to be connected to a round bottom flask within the microwave oven. Boiling chips can be employed in lieu of a stir bar to help reduce uneven superheating of solvent or bumping, and agitation resulting from solvent boiling creates a stirring effect.

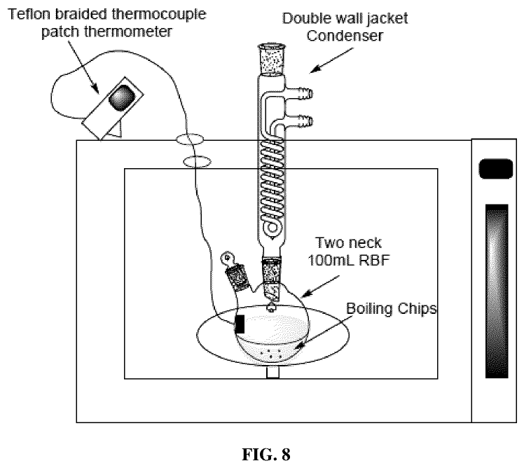

FIGS. 9A-9C are HPLC traces of a reaction mixture samples from a microwave-assisted continuous flow reactor measured at 0 minutes (FIG. 9A), 2.5 minutes (FIG. 9B), and 7.5 minutes (FIG. 9C).

FIG. 10 is a diagram depicting a sonication-assisted continuous flow reactor.

FIGS. 11A-11C are HPLC traces of a reaction mixture samples from a sonication-assisted continuous flow reactor measured at 5 minutes (FIG. 11A), 10 minutes (FIG. 11B), and 15 minutes (FIG. 11C).

FIG. 12 is a diagram depicting a continuous flow reactor that can be used to produce hexahydrocannabinol from Δ-8 tetrahydrocannabinol, Δ-9 tetrahydrocannabinol, or a mixture thereof.

FIG. 13 is a diagram depicting a reaction scheme for producing hexahydrocannabinol from Δ-8 tetrahydrocannabinol, Δ-9 tetrahydrocannabinol, Δ-10 tetrahydrocannabinol, exo-tetrahydrocannabinol, or a mixture thereof.

FIG. 14 is a diagram depicting the conversion of THC to HHC over time, as produced by a room temperature continuous flow reactor.

FIG. 15 is an HPLC trace measured at 540 minutes of a product produced by a continuous flow reactor.

FIG. 16 is a diagram depicting a bifurcated continuous flow reactor for producing hexahydrocannabinol from cannabidiol.

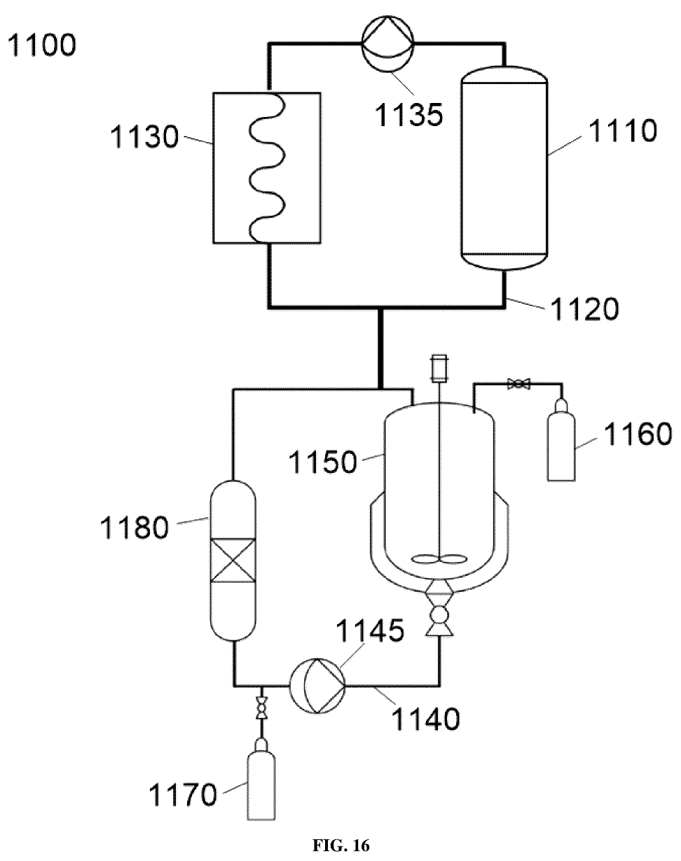

FIG. 17 is a table that includes relative amounts of products of microwave-assisted CBD acid-catalyzed cyclization reactions performed under different solvent/acid combinations.

FIG. 18 is a table that includes relative amounts of products of sonicator-assisted CBD acid-catalyzed cyclization reactions performed under different solvent/acid combinations.

### DETAILED DESCRIPTION OF THE INVENTION

Flow chemistry is a technology that provides access to chemical processes that are otherwise inefficient or problematic. A flow chemistry module essentially establishes a stable set of conditions through which reagents are passed. The key advantages of flow chemistry, including correlation between reaction time and position within the reactor and ease of replication, allow for a flow module to be tailored and adapted to provide, high and reproducible yields. Additionally, flow conditions could provide short dispersal paths and improved mass transference rates, resulting in better yields and selectivity.

Microwave irradiation is a technique that has become more prevalent in recent years in chemical syntheses. The traditional method of heating heats the reaction mixture from the outside in, which can cause inconsistent reaction kinetics, and uneven heating which can necessitate stirring to distribute heat evenly. Heating with microwave irradiation is a direct method to heat a reactor from inside when employing microwave-transparent reaction vessels. The microwave radiation provides deeper penetration and heats the solvent and sample evenly while maintaining consistent reaction kinetics.

Combining microwave irradiation and synthesis with flow reactors can be advantageous because microwave irradiation allows one to create heat inside continuous-flow devices at locations where interactions between two phases occur. Microwave irradiation can be used to address kinetic problems when relatively narrow flow channels create solid phase/solution phase mixing problems. The combination of microwave irradiation and flow reactors can also be useful because microwave chemistry can be difficult to scale-up in batch environments.

Reductions are generally performed using gaseous hydrogen or soluble reductants that serve as hydride sources. Safety and control over reductions is much better under flow conditions than under larger, batch conditions. Hydrogenations generally require high pressures of a highly flammable gas and oftentimes employ pyrophoric catalysts. Liquid reductants, such as hydride sources, often face selectivity issues, as products may be susceptible to over-reduction. These issues can be reproducibly addressed using flow conditions. Hydrogenations are well-suited for flow conditions due to the facility of pressurization and enhanced safety.

An alternative approach to hydrogen introduction in a flow system is in situ generation, which both minimizes equipment and maximizes safety. In some aspects, an electrolytic cell can be used in conjunction with a continuous flow reactor in order to generate hydrogen gas in situ. The rate of water electrolysis and hydrogen gas production can be tailored to match other flow conditions, such as molar flow rate through a given cross-sectional area.

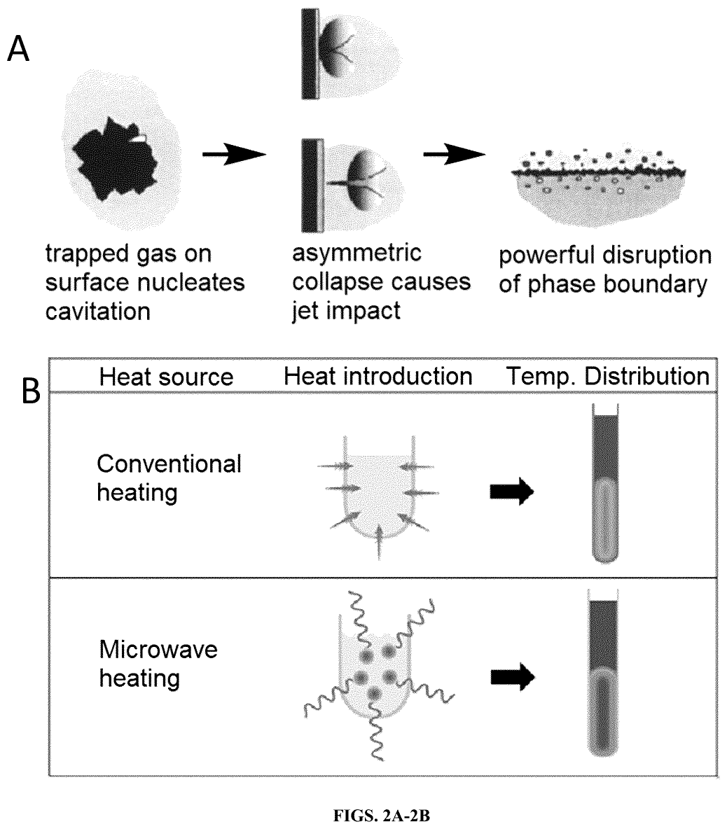

Integrating ultrasound or sonication with flow reactors can not only be used to add energy to a reaction mixture, but also to address clogging and mixing issues. Sonication addresses kinetic problems by producing cavitation bubbles, where high temperatures and pressures are experienced within the microenvironment of the cavitation bubbles produced (FIG. 2A). As the ultrasound intensity increases, the reaction rate increases due to an increase in the number of cavitation bubbles and an increase in the temperature within the cavitation bubbles.

In batch and large-scale reactors, sonication has been widely used to intensify mixing, mass transfer, and reaction rates. However, it is considered difficult to control and scale, due to non-uniformly generated acoustic fields and the complex flow patterns within conventional reactors. Flow reactors offer a solution to these issues because the size range of ultrasonic effects are within the size range of that of the flow channels. Incorporating sonication within flow reactors can be advantageous by expediting reaction kinetics and creating reactive microenvironments around the sonicating field, thereby providing a greener way of producing products in comparison to standard bench reactions.

For a continuous flow reactor, the reactor channel diameters can be selected to provide an internal diameter that facilitates mixing and back pressure properties. For example, a smaller diameter reactor channel can be selected to increase shear. The internal diameters of the reactor channels disclosed herein can be passage or chamber may be any one of, less than, greater than, or between 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 mm. The internal diameter of reactor channels can also vary throughout the flow reactor. Different internal diameters will affect the fluid flow within the reactor, and the internal diameters can be selected to address increases in viscosity, introduction of reactants, creation of solid precipitates, changes in internal pressure, and the like.

### I. CHEMICAL DEFINITIONS

The terms delta-8 tetrahydrocannabinol, delta-8 THC, D8 THC, D8, Δ8-tetrahydrocannabinol, and Δ8-THC are used interchangeably herein. The terms delta-9 tetrahydrocannabinol, delta-9 THC, D9 THC, D9, Δ9-tetrahydrocannabinol, Δ9-THC, and THC are used interchangeably herein. The terms delta-10 tetrahydrocannabinol, delta-10 THC, D10 THC, D10, Δ10-tetrahydrocannabinol, and Δ10-THC are used interchangeably herein. The terms exo-tetrahydrocannabinol and exo-THC are used interchangeably herein. The terms hexahydrocannabinol and HHC are used interchangeably herein.

The term “alkyl” includes straight-chain alkyl, branched-chain alkyl, cycloalkyl (alicyclic), heteroatom-unsubstituted alkyl, heteroatom-substituted alkyl, heteroatom-unsubstituted Cn-alkyl, and heteroatom-substituted Cn-alkyl. In certain embodiments, lower alkyls are contemplated. The term “lower alkyl” refers to alkyls of 1-6 carbon atoms (that is, 1, 2, 3, 4, 5 or 6 carbon atoms). The term “heteroatom-unsubstituted Cn-alkyl” refers to a radical, having a linear or branched, cyclic or acyclic structure, further having no carbon-carbon double or triple bonds, further having a total of n carbon atoms, all of which are nonaromatic, 3 or more hydrogen atoms, and no heteroatoms. For example, a heteroatom-unsubstituted C₁-C₁₀-alkyl has 1 to 10 carbon atoms. The groups, —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), CH₂CH(CH₃)₂ (iso-butyl), —C(CH₃)₃ (tent-butyl), —CH₂C(CH₃)₃ (neo-pentyl), cyclobutyl, cyclopentyl, and cyclohexyl, are all non-limiting examples of heteroatom-unsubstituted alkyl groups. The term “heteroatom-substituted Cn-alkyl” refers to a radical, having a single saturated carbon atom as the point of attachment, no carbon-carbon double or triple bonds, further having a linear or branched, cyclic or acyclic structure, further having a total of n carbon atoms, all of which are nonaromatic, 0, 1, or more than one hydrogen atom, at least one heteroatom, wherein each heteroatom is independently selected from the group consisting of N, O, F, Cl, Br, I, Si, P, and S. For example, a heteroatom-substituted C₁-C₁₀-alkyl has 1 to 10 carbon atoms. The following groups are all non-limiting examples of heteroatom-substituted alkyl groups: trifluoromethyl, —CH₂F, —CH₂Cl, —CH₂Br, piperidinyl, —CH₂OH, —CH₂OCH₃, —CH₂OCH₂CF₃, —CH₂OC(O)CH₃, —CH₂NH₂, —CH₂NHCH₃, —CH₂N(CH₃)₂, —CH₂CH₂Cl, —CH₂CH₂OH, CH₂CH₂OC(O)CH₃, —CH₂CH₂NHCO₂C(CH₃)₃, and —CH₂Si(CH₃)₃.

The term “alkenyl” includes straight-chain alkenyl, branched-chain alkenyl, cycloalkenyl, cyclic alkenyl, heteroatom-unsubstituted alkenyl, heteroatom-substituted alkenyl, heteroatom-unsubstituted Cn-alkenyl, and heteroatom-substituted Cn-alkenyl. In certain embodiments, lower alkenyls are contemplated. The term “lower alkenyl” refers to alkenyls of 1-6 carbon atoms (that is, 1, 2, 3, 4, 5 or 6 carbon atoms). The term “heteroatom-unsubstituted Cn-alkenyl” refers to a radical, having a linear or branched, cyclic or acyclic structure, further having at least one nonaromatic carbon-carbon double bond, but no carbon-carbon triple bonds, a total of n carbon atoms, three or more hydrogen atoms, and no heteroatoms. For example, a heteroatom-unsubstituted C₂-C₁₀-alkenyl has 2 to 10 carbon atoms. Heteroatom-unsubstituted alkenyl groups include: —CH═CH₂ (vinyl), —CH═CHCH₃, —CH═CHCH₂CH₃, —CH₂CH═CH₂ (allyl), —CH₂CH═CHCH₃, and —CH═CH—C₆H₅. The term “heteroatom-substituted Cn-alkenyl” refers to a radical, having a single nonaromatic carbon atom as the point of attachment and at least one nonaromatic carbon-carbon double bond, but no carbon-carbon triple bonds, further having a linear or branched, cyclic or acyclic structure, further having a total of n carbon atoms, 0, 1, or more than one hydrogen atom, and at least one heteroatom, wherein each heteroatom is independently selected from the group consisting of N, O, F, Cl, Br, I, Si, P, and S. For example, a heteroatom-substituted C₂-C₁₀-alkenyl has 2 to 10 carbon atoms. The groups, dihydrofuranyl, —CH═CHF, —CH═CHCl and —CH═CHBr, are non-limiting examples of heteroatom-substituted alkenyl groups. In some embodiments, a continuous flow process as disclosed herein is employed to reduce an alkenyl group and provide the corresponding alkyl group.

Unless stated to the contrary, a formula with chemical bonds shown only as solid lines and not as wedges or dashed lines contemplates each possible isomer, e.g., each enantiomer, diastereomer, and meso compound, and a mixture of isomers, such as a racemic or scalemic mixture.

The claimed invention is also intended to encompass salts of any of the compounds of the present invention. The term “salt(s)” as used herein, is understood as being acidic and/or basic salts formed with inorganic and/or organic acids and bases. Zwitterions (internal or inner salts) are understood as being included within the term “salt(s)” as used herein, as are quaternary ammonium salts such as alkylammonium salts. Nontoxic, pharmaceutically acceptable salts are preferred, although other salts may be useful, as for example in isolation or purification steps during synthesis. Salts include, but are not limited to, sodium, lithium, potassium, amines, tartrates, citrates, hydrohalides, phosphates and the like. A salt may be a pharmaceutically acceptable salt, for example. Thus, pharmaceutically acceptable salts of compounds of the present invention are contemplated.

The term “pharmaceutically acceptable salts,” as used herein, refers to salts of compounds of this invention that are substantially non-toxic to living organisms. Typical pharmaceutically acceptable salts include those salts prepared by reaction of a compound of this invention with an inorganic or organic acid, or an organic base, depending on the substituents present on the compounds of the invention.

Compounds employed in methods of the invention may contain one or more asymmetrically-substituted carbon or nitrogen atoms, and may be isolated in optically active or racemic form. Thus, all chiral, diastereomeric, racemic form, epimeric form, and all geometric isomeric forms of a structure are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present invention can have the S- or the R-configuration, as defined by the IUPAC 1974 Recommendations. Compounds may be of the D- or L-form, for example. It is well known in the art how to prepare and isolate such optically active forms. For example, mixtures of stereoisomers may be separated by standard techniques including, but not limited to, resolution of racemic form, normal, reverse-phase, and chiral chromatography, preferential salt formation, recrystallization, and the like, or by chiral synthesis either from chiral starting materials or by deliberate synthesis of target chiral centers.

In addition, atoms making up the compounds of the present invention are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

Various forms of palladium catalyst useful for the reaction are discussed by Blaser et. al., Supported palladium catalysts for fine chemicals synthesis in Journal of Molecular Catalysis A: Chemical, 2001, v. 172, p. 3-18, the entirety of which is incorporated by reference.

### II. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. Example 1—Synthesis of Tetrahydrocannabinol Using Microwave-Assisted Flow Conditions

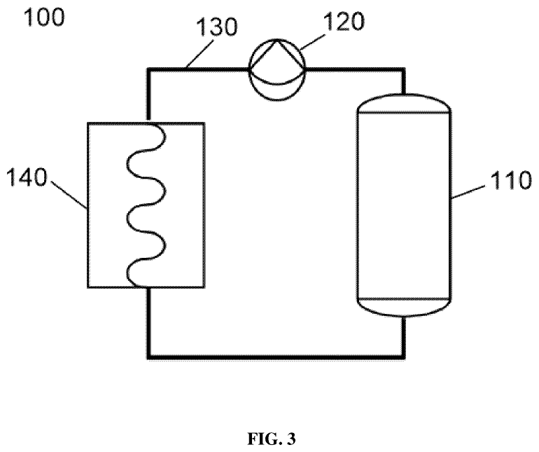

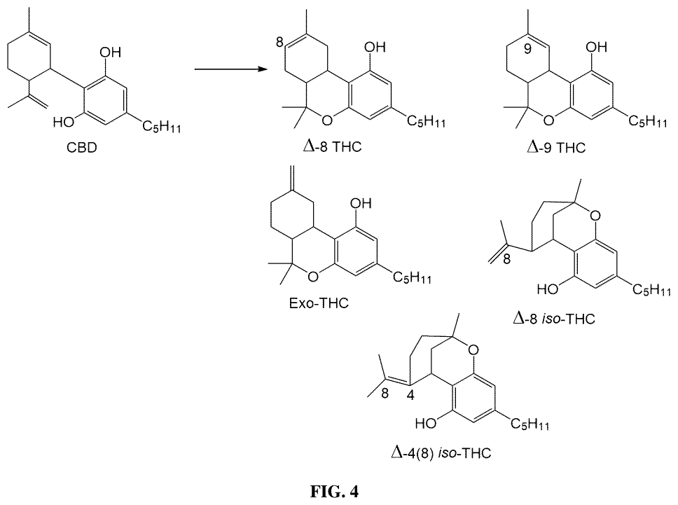

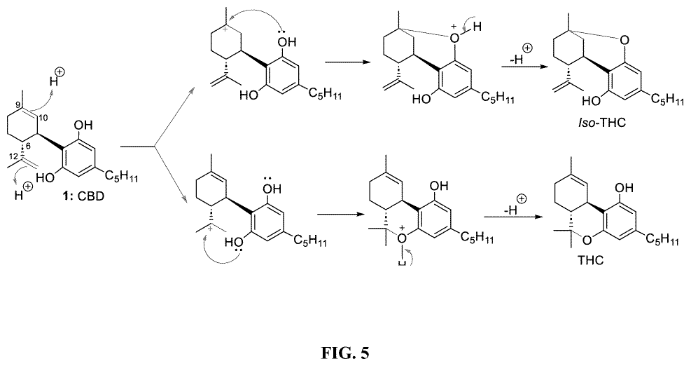

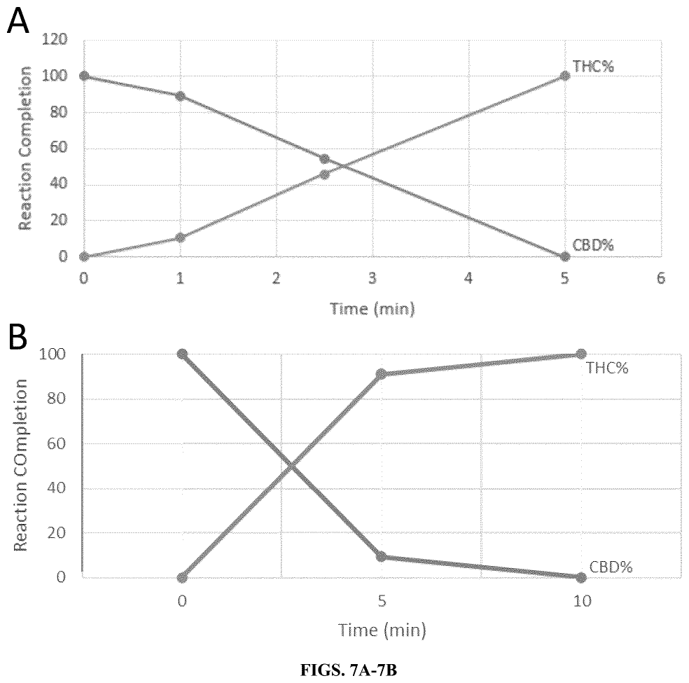

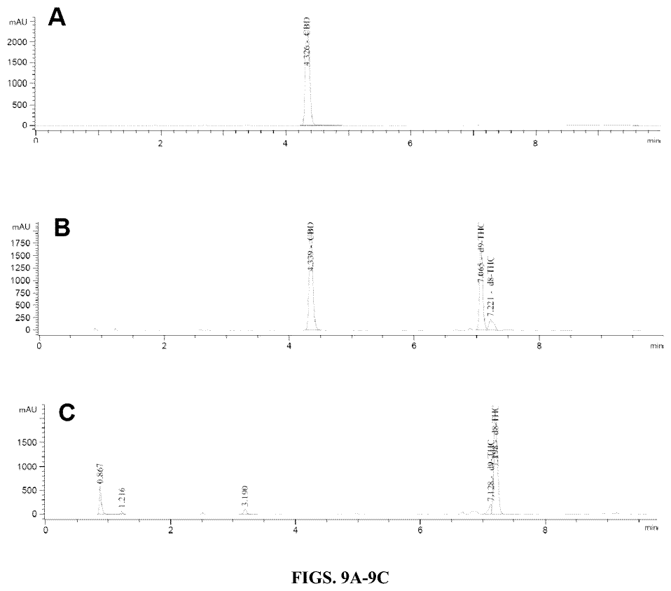

A microwave-assisted continuous flow reactor 100 (FIG. 3) was used to synthesize a mixture of Δ-8 tetrahydrocannabinol and Δ-9 tetrahydrocannabinol from cannabidiol. The two products are depicted in FIG. 4, along with other cyclization side-products. Mechanisms for the transformation of cannabidiol into Δ-9 tetrahydrocannabinol and side product iso-tetrahydrocannabinol are depicted in FIG. 5. CBD (50 g) was dissolved in hexanes (100 mL) in holding tank 110. A Lewis or Bronsted acid was added to the solution. A diaphragm pump 120 was utilized to push the reaction mixture through flow path 130 into a 1,200 watt microwave reactor 140 and back into holding tank 110. After 5 mins, the reaction reached completion by HPLC showing 100% consumption of CBD and producing a THC yield of 82%, prior to purification. A plot of the reaction progress over time is depicted in FIG. 7A, where the THC data points indicate combined amounts of Δ-8 tetrahydrocannabinol and Δ-9 tetrahydrocannabinol produced. The HPLC traces in FIGS. 9A-9C show the reaction progress at t=0 minutes (FIG. 9A), t=2.5 minutes (FIG. 9B), and t=7.5 minutes (FIG. 9C). The HPLC peak at approximately 4.2 minutes corresponds to cannabidiol, and the two HPLC peaks between 7 and 7.5 minutes correspond to Δ-8 tetrahydrocannabinol and Δ-9 tetrahydrocannabinol.

### B. Example 2—Synthesis of Tetrahydrocannabinol Using Sonicator-Assisted Flow Conditions

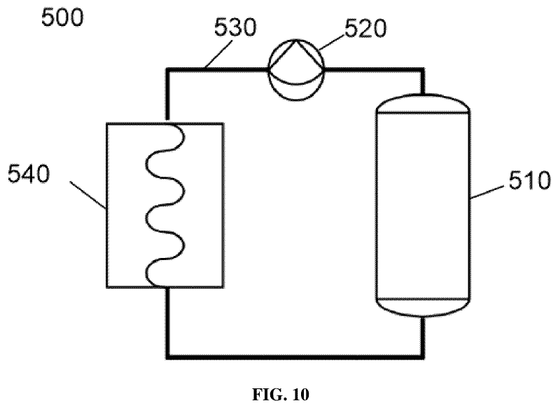

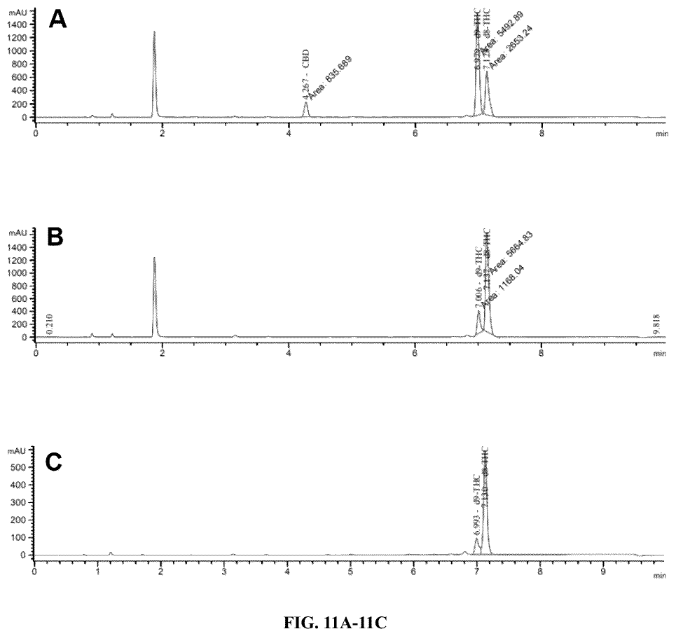

A sonicator-assisted continuous flow reactor 500 (FIG. 10) was used to synthesize a mixture of Δ-8 tetrahydrocannabinol and Δ-9 tetrahydrocannabinol from cannabidiol. The two products are depicted in FIG. 4, along with other cyclization side-products. CBD (6 kg) was dissolved in toluene (12 L) in holding tank 510. A Lewis or Bronsted acid was added to the solution. A diaphragm pump 520 was utilized to push the reaction mixture through flow path 530 into sonicator bath 540 and back into holding tank 510. The reaction was run for 10 minutes. After 10 mins, the reaction reached completion by HPLC showing 100% consumption of CBD and producing a THC yield of 85%, prior to purification. The HPLC traces in FIGS. 11A-11C show the reaction progress at 5 minutes (FIG. 11A), 10 minutes (FIG. 11B), and 15 minutes (FIG. 11C). The HPLC peak at approximately 4.2 minutes corresponds to cannabidiol, and the two HPLC peaks between around 6.9 and 7.3 minutes correspond to Δ-8 tetrahydrocannabinol and Δ-9 tetrahydrocannabinol.

### C. Example 3—Scaled Synthesis of Tetrahydrocannabinol Using Microwave-Assisted Flow Conditions

A microwave-assisted continuous flow reactor was used to synthesize a mixture of Δ8-tetrahydrocannabinol and Δ9-tetrahydrocannabinol from cannabidiol. Cannabidiol (500 g, 1.6 mol) was dissolved in hexanes (1 L) to obtain a 1.6 M solution in a holding tank. p-Toluenesulfonic acid (15.22 g, 0.008 mol) was added to the solution. A diaphragm pump was utilized to push the reaction mixture through the flow path into a 1200 W microwave reactor and back into the holding tank. The reaction reached completion after 5 min. Reaction monitoring by HPLC showed that CBD had been completely consumed, and that an 82% total THC yield had been achieved, prior to purification.

¹H NMR (500 MHz, CDCl₃) δ 6.33 (d, J=1.6 Hz, 1H), 6.12 (d, J=1.6 Hz, 1H), 5.53 (s, 1H), 5.49-5.44 (m, 1H), 3.28 (dd, J=16.4, 4.7 Hz, 1H), 2.76 (td, J=10.9, 4.5 Hz, 1H), 2.44 (td, J=7.5, 2.9 Hz, 3H), 2.18 (ddt, J=11.5, 5.5, 2.8 Hz, 1H), 1.88-1.82 (m, 2H), 1.56 (dq, J=14.2, 7.2 Hz, 3H), 1.43 (s, 3H), 1.33 (qq, J=7.4, 4.3 Hz, 5H), 1.14 (s, 3H), 0.96-0.87 (m, 4H).

### D. Example 4—Scaled Synthesis of Δ9THC and Δ8THC Using Sonication-Assisted Flow Conditions

A sonication-assisted continuous flow reactor 500 (FIG. 10) was used to synthesize a mixture of Δ8-tetrahydrocannabinol and Δ9-tetrahydrocannabinol from cannabidiol. Cannabidiol (6000 g, 19.11 mol) was dissolved in toluene (12 L) to obtain a 1.6 M solution in a holding tank. p-Toluenesulfonic acid (181.53 g, 0.96 mol) was added to the solution. A diaphragm pump was utilized to push the reaction mixture through the flow path into the ultrasound bath, which was heated at 60° C. and flowed back into the holding tank. The reaction was run for 10 min at room temperature with final reaction temperature at 60° C. The reaction reached completion after 20 min; the HPLC results showed that CBD had been completely consumed and that an 85% total THC yield had been achieved.

¹H NMR (500 MHz, CDCl₃) δ 6.33 (d, J=1.6 Hz, 1H), 6.12 (d, J=1.6 Hz, 1H), 5.53 (s, 1H), 5.49-5.44 (m, 1H), 3.28 (dd, J=16.4, 4.7 Hz, 1H), 2.76 (td, J=10.9, 4.5 Hz, 1H), 2.44 (td, J=7.5, 2.9 Hz, 3H), 2.18 (ddt, J=11.5, 5.5, 2.8 Hz, 1H), 1.88-1.82 (m, 2H), 1.56 (dq, J=14.2, 7.2 Hz, 3H), 1.43 (s, 3H), 1.33 (qq, J=7.4, 4.3 Hz, 5H), 1.14 (s, 3H), 0.96-0.87 (m, 4H).

### C. Example 5—Synthesis of Hexahydrocannabinol

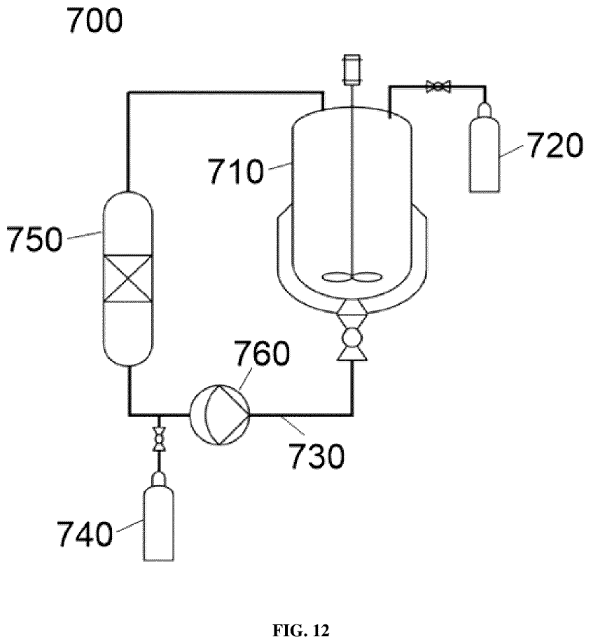

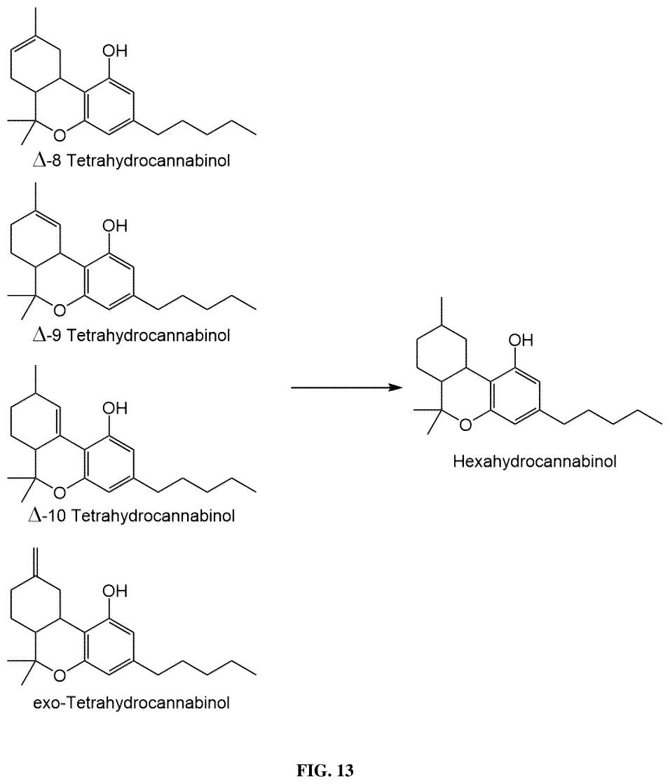

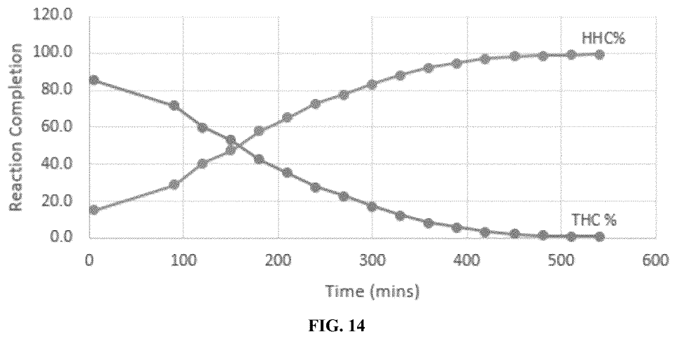

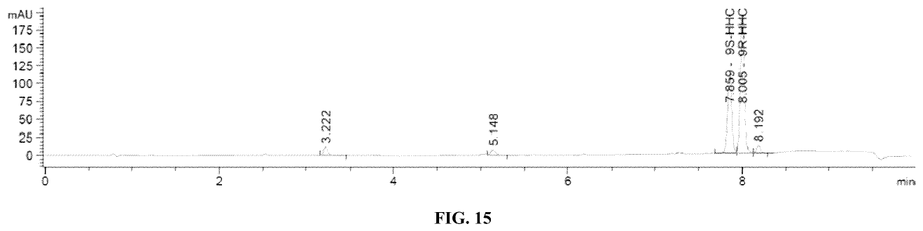

A continuous flow reactor 700 (FIG. 12) was used to synthesize hexahydrocannabinol from a mixture of Δ-8 tetrahydrocannabinol and Δ-9 tetrahydrocannabinol. The reaction scheme in FIG. 13 shows different tetrahydrocannabinol starting materials (Δ-8 tetrahydrocannabinol, Δ-9 tetrahydrocannabinol, Δ-10 tetrahydrocannabinol, and exo-tetrahydrocannabinol) that can be converted to hexahydrocannabinol by reduction of the cyclohexyl olefin. Reservoir 710 was placed under vacuum then filled with argon gas from inert gas reservoir 720. A mixture of Δ-8 tetrahydrocannabinol and Δ-9 tetrahydrocannabinol (40 kg) and ethanol (75 L) was added to reservoir 710. Candle filter 750 was loaded with 7.5 kg of palladium on carbon catalyst. The reaction solution of Δ-8 tetrahydrocannabinol and Δ-9 tetrahydrocannabinol in ethanol was then pumped through reactor loop 730. A carb stone was used to introduce hydrogen gas from hydrogen gas reservoir 740 into reactor loop 730. The reaction was run at room temperature by pumping the reaction solution through reaction loop 730. After 9 hours, the reaction progress was monitored by HPLC. A plot of the reaction progress over time is depicted in FIG. 14. The 9-hour timepoint HPLC trace (FIG. 15) showed that the reaction mixture contained 99.2% hexahydrocannabinol and <1% tetrahydrocannabinols.

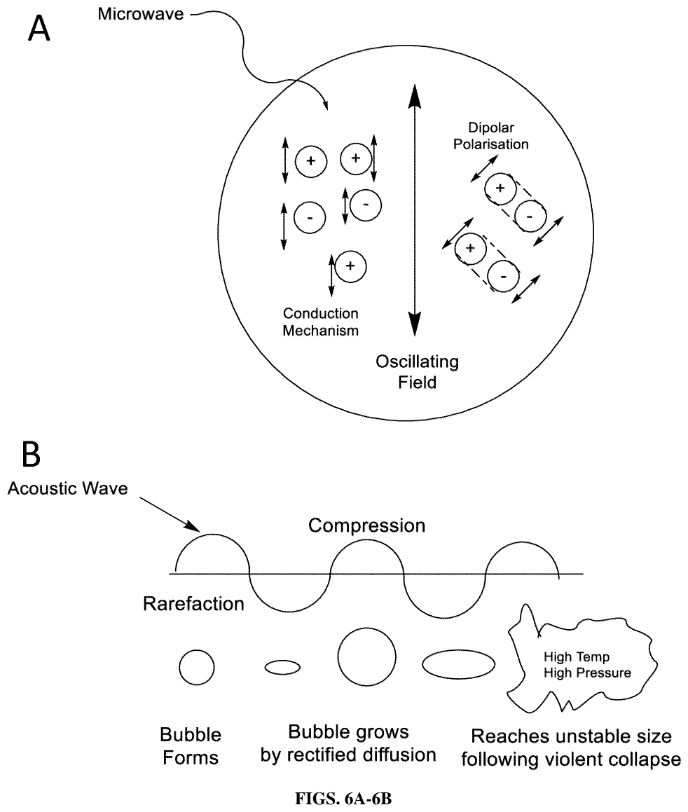

Acid-promoted cyclization of cannabidiol occurs via activation of a specific double bond (FIG. 5). The cannabidiol scaffold has two exocyclic double bonds, one in the cyclohexyl ring (Δ9) and another in the propenyl chain (Δ12). When the activation occurs on Δ12, the chromene ring forms to afford the THC scaffold (FIG. 5, bottom). Moreover, activation at the Δ9 double bond turns the cyclization toward oxocine ring formation to furnish Δ8-iso-THC (FIG. 5, top). The chromene ring is bonded linearly between the resorcinol and the terpenyl moiety, whereas the oxocin ring is “connected”. Therefore, the tetrahydrocannabinol-core can be the presumed to be the major product because it is more energetically stable. Several refined methodologies to produce THC have been reported using acid catalysis or various synthetic pathways. These conventional synthetic approaches describe the formation of not only Δ-8 tetrahydrocannabinol and Δ-9 tetrahydrocannabinol isomers, but also around 3-15% of Δ8-iso-THC. The use of microwave and sonication was observed to increase the reaction rates either through direct intervention through heating and polarization of reactants and solvent (FIG. 6A), or through acoustic manipulation such as causing cavitation bubbles (FIG. 6B) creating microenvironments of pressure and temperature differences. Due to the short reaction times with noticeable conversions, microwave and sonication are observed to provide commensalistic benefits by decreasing reaction times and increasing conversions with commonly used acids.

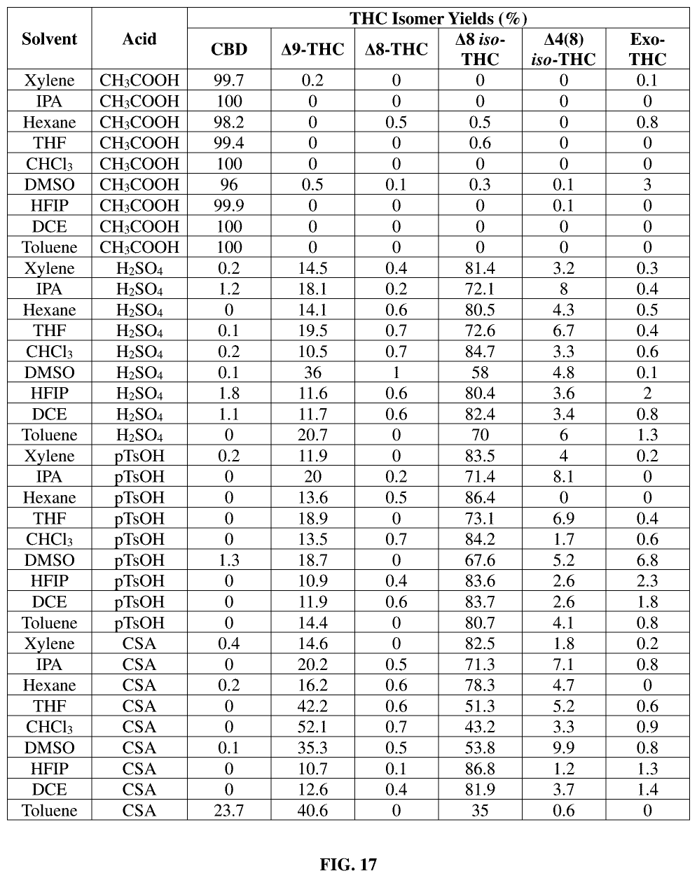

To examine the selectivity of CBD cyclization, diverse reactions involving the use of organic and inorganic acids in various solvents were accomplished using microwave energy (FIG. 17). The reactions were carried out in a modified commercial microwave oven using an open-vessel technique with modified temperature monitoring. Microwave reaction conditions: 0.5 g of CBD, 1.5 mL of solvent, 5% catalytic amount of acid, MW (1200 W), 5 min reaction time. The percentage ratios of the THC isomers were determined by using HPLC. The temperature baseline was 110° C. as the original scaled flow setup was done at this temperature for a constant reaction time.

The results indicate that acetic acid (CH₃COOH) is not a good catalyst for the cyclization of cannabidiol. Although reaction times generally took 2 min, the reactions were microwaved for a total of 5 min in an effort to influence conversion, and almost 100% of CBD was recovered in all cases. Using sulfuric acid as the catalyst, Δ8-THC and Δ9-THC were afforded as the main products, but other THC isomers were also obtained (FIG. 17, entries 10-18). Δ4(8)-iso-THC was accomplished with 8.0, 6.7, and 6.0% when the reaction was carried out in isopropanol (IPA), tetrahydrofuran (THF), or toluene as the solvent, respectively (FIG. 17, entries 11, 13, and 18). pTsOH in hexane converted CBD into Δ8- and Δ9-THC mixtures in a better yield than sulfuric acid and CSA with no isomer formation (FIG. 17, entry 21). The use of other solvents performed different grades of regioselectivity. Camphor sulfonic acid (CSA) arises as an attractive cyclization inducer because, in combination with hexafluoro-2-propanol (HFIP), a polar, strongly hydrogen-bond-donating solvent, the regioselectivity of the CBD cyclization moved toward the generation of Δ8-THC with an 86.8% yield (FIG. 17, entry 34). The combination of CSA as the catalyst and toluene as the solvent did not complete the reaction, obtaining 23.7% CBD with 40.6 and 35.0% of Δ9- and Δ8-THC, respectively (FIG. 17, entry 36). The effects of modifying microwave power in the conversion of CBD into THC isomers using pTsOH in hexane was then examined (Table 1).

TABLE 1
Effect of Microwave Power on Microwave-
Assisted CBD Acid-Catalyzed Cyclization*

| | THC Isomer Yields (%) | | | | | |
|---|---|---|---|---|---|---|
| Microwave Power | | Δ9- | Δ8- | Δ8 iso- | Δ4(8) | Exo- |
| (W) | CBD | THC | THC | THC | iso-THC | THC |
| 240 | 97.5 | 2.5 | 0 | 0 | 0 | 0 |
| 480 | 83.1 | 15.1 | 1.8 | 0 | 0 | 0 |
| 720 | 6.2 | 46.1 | 47.7 | 0 | 0 | 0 |
| 960 | 0 | 28.8 | 71.2 | 0 | 0 | 0 |
| 1,200 | 0 | 13.6 | 86.4 | 0 | 0 | 0 |

*Reaction conditions - 0.5 g of CBD, 1.5 mL of hexane, 5% catalytic mount of pTsOH, MW, 5 min reaction time.

The results show that microwave power can affect the heating rate during the reaction and therefore influences the formation ratios of THC isomers during the cyclization of CBD. When performing the reaction at a setup microwave power of 240 W, only 2.5% of Δ9-THC was afforded (Table 1, entry 1). When the microwave power is increased, the CBD conversion is higher and increases the ratio of Δ8-THC. At 960 W, CBD fully converted into Δ9- and Δ8-THC with ratios of 28.8 and 71.2%, respectively (Table 1, entry 4). These outcomes prove the premise that Δ8-THC is thermodynamically more stable than its isomer Δ9-THC.

The best conditions found for the conversion of CBD in a mixture of Δ8THC and Δ9THC (pTsOH and hexane: FIG. 17, entry 19) were applied to a continuous flow chemistry system assisted by microwave. The reaction comprised adding pTsOH to a CBD solution in hexane and feeding the reaction mixture through a continuous flow loop, which is inside the microwave reactor as an energizing component. The reaction was complete within 5 min and yielded mixtures of 91 and 9% Δ8-THC and Δ9-THC, respectively, as determined by HPLC. The treatment of this reaction in a continuous flow system enables more accurate management of reaction factors, and is more suitable for optimization and scale-up.

To prove that the technique established for CBD cyclization is not restricted only to a small scale, this reaction was scaled up to develop a procedure to produce THC in kilogram quantities. A microwave-assisted continuous flow reactor was designed (FIG. 3) to carry out the conversion of 500 g of CBD into THC catalyzed by hexane as the solvent. The reaction was completed after 5 min and afforded an 82% yield of Δ8-THC and Δ9-THC as an isolated mixture (94:6 regioselectivity ratio); the reaction graph generated is shown in FIG. 3A.

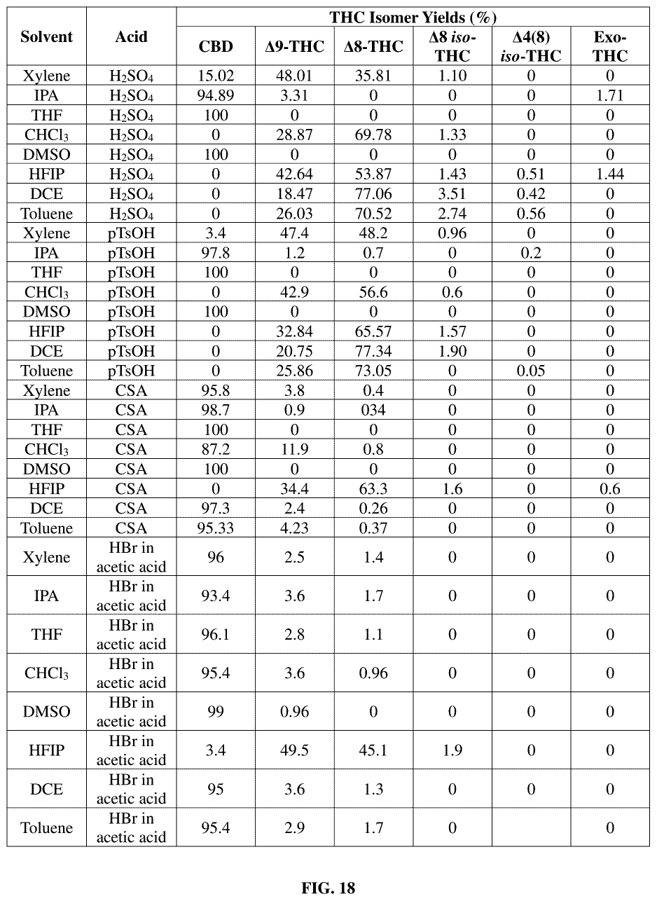

An ultrasound with flow reactor was employed to promote the acid-cyclization of CBD using pTsOH in the presence of hexane (FIG. 10). This appears to be the first time that sonication has been merged with flow chemistry for the synthesis of THC. Results from the use of sonication to influence the acid cyclization of CBD were varied (FIG. 18). Sonication Reaction conditions: 0.5 g of CBD, 1.5 mL of solvent, 5% catalytic amount of acid, sonication frequency 40 kHz, 2 min reaction time. The temperature baseline was 60° C. The use of acetic acid within the microwave proved to be inefficient within the cyclization (FIG. 17). 33% HBr in acetic acid was used in the sonication trial as shown in FIG. 18. CBD conversion remained low with minute amounts of THC or isomers being created. The use of HFIP as a solvent produced an almost 1:1 ratio of Δ9-THC and Δ8-THC with some CBD still unconverted. Use of other polar and nonpolar solvents did not contribute to cyclization. The use of camphor sulfonic acid (CSA) in various solvents did not produce the desired product except HFIP where less than 1% CBD was detected and Δ8-THC and Δ9-THC were detected with minimal unwanted isomer formation. Xylene, IPA, THF, and DMSO were the solvents that did not convert CBD to THC. Xylene did convert the CBD, but a detectable amount of CBD remained within the reaction. The use of H₂SO₄ in DMSO, IPA, and xylene had a detectable amount of unconverted CBD; although in xylene, Δ8-THC and Δ9-THC were produced, the conversion was lower than in CHCl₃, HFIP, DCE, and toluene solvents. Although the results differ from those of the microwave, the sonicator had a stable 60° C. temperature, and did not have various heating spots or localized superheating spots, as would be produced with the microwave.

Similar to the microwave scaled reaction, a scaled sonicator flow reactor was created, and toluene with pTsOH were used. A large-scale sonication-assisted continuous flow reactor was designed to carry out the conversion of 6,000 g of CBD into THC, using pTsOH as the acid (5%) and toluene as the solvent heated at 60° C. The schematic in FIG. 10 represents the reactor design that was used to accomplish the acid-cyclization of CBD (6 kg) inside of the ultrasound bath cavity using a continuous flow process technique. CBD was completely converted to Δ8-THC and Δ9-THC after 10 min (83:17 regioselectivity ratio), with 85% isolated yield.

The sonication flow chemistry technique, when performed at room temperature, higher Δ9-THC:Δ8-THC ratios and fewer unwanted isomers were observed, as compared to results obtained from the microwave-assisted flow chemistry technique. When higher temperatures were employed in the microwave flow chemistry experiments, isomerization of Δ9-THC into Δ8-THC was observed, as Δ8-THC is the more thermodynamically stable isomer.

The microwave and sonication-assisted flow processes demonstrated the ability to scale past milligram and gram scales and afforded mixture of isomers. These isomeric mixtures are ideal feedstock for hydrogenation reactions to produce hexahydrocannabinol.

In summary, the present inventors have developed unique reactor systems and techniques for producing cannabinoids in large quantities with decreased reaction times using the combination of flow conditions with microwave and ultrasound reactors. The flow chemistry processes disclosed herein enable a high degree of precision in the delivery of reagents and solutions, and excellent control over the conditions to which the solutions are exposed. Precise control results in excellent reproducibility and safety, thereby making these methods amenable to larger scales. The processes disclosed herein can also be used in tandem, thereby providing a means to perform multistep syntheses under flow conditions. The continuous flow processes can be used to produce the desired products at the kilogram scale within minutes. Quick and clean production of cannabinoids is important within the industrial sector, but also within the pharmaceutical sector, as research into cannabinoids and their analogues for uses in treatment of diseases and cancers becomes more prevalent.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A continuous flow process for producing tetrahydrocannabinol from a cannabidiol starting material, the process comprising:
mixing a cannabidiol starting material with a solvent to form a solution;
adding an acid to the solution to form a reaction solution;
feeding the reaction solution through a continuous flow loop; and
passing the reaction solution through an energizing component to produce tetrahydrocannabinol.

2. The process of claim 1, wherein the energizing component is in-line with the continuous flow loop.

3. The process of claim 1, wherein the energizing component is a microwave reactor, a sonicator, a heating bath, or a combination thereof.

4. The process of claim 1, wherein when the energizing component comprises a microwave reactor, at least a portion of the continuous flow loop is microwave-transparent.

5. The process of claim 1, wherein when the energizing component comprises a heating bath, the process further comprises heating the reaction solution to a temperature ranging from 25° C. to 100° C.

6. The process of claim 1, further comprising passing the reaction through the continuous flow loop for a period of time ranging from 2 minutes to 20 minutes.

7. The process of claim 1, wherein at least a portion of a flow path within the energizing component is non-linear.

8. The process of claim 7, wherein the a flow path within the energizing component is coiled.

9. The process of claim 1, wherein the solvent is selected from the group consisting of methanol, ethanol, propanol, isopropanol, butanol, tert-butanol, THF, 2-Me-THF, toluene, and ethyl acetate.

10. The process of claim 1, wherein the tetrahydrocannabinol comprises delta-8 tetrahydrocannabinol, delta-9 tetrahydrocannabinol, or a combination thereof.

11. A continuous flow process for producing hexahydrocannabinol from a tetrahydrocannabinol starting material, the process comprising:
mixing a tetrahydrocannabinol starting material with a solvent to form a first solution;
feeding the first solution into a continuous flow loop;
feeding hydrogen gas into the continuous flow loop to form a combined feed comprising the hydrogen gas and the first solution; and
passing the combined feed over a catalyst bed within the continuous flow loop to produce hexahydrocannabinol.

12. The process of claim 11, wherein the tetrahydrocannabinol starting material is delta-8 tetrahydrocannabinol, delta-9 tetrahydrocannabinol, delta-10 tetrahydrocannabinol, exo-tetrahydrocannabinol, or a mixture thereof.

13. The process of claim 11, wherein the catalyst bed is provided on a candle filter, Nutsche filter, ZWAG filter, or FUNDA filter support.

14. The process of claim 11, wherein the catalyst is selected from the group consisting of Pd/C, Pt/C, Rh/C, Ru/C, Ir/C, Ni/C, Fe/C, Co/C, V/C, Mn/C, Raney nickel, Raney cobalt, Pd/alumina, Pt/alumina, Pt/activated charcoal, Pt₂O (Adam's catalyst), Wilkinson's catalyst ([RhCl(PPh₃)₃]), Crabtree's catalyst ([C₈H₁₂IrP(C₆H₁₁)₃C₅H₅N]PF₆), 9-borabicyclo[3.3.1]nonane, alpine borane, BH₃-DMSO, BH₃-THF, and N-methylimidodiacetic (MIDA) boronates, tetrakis(triphenylphospine)palladium, metal on alumina, metal on activated charcoal including Pd/activated charcoal, metal oxides, metal hydroxides, metal salts, metal halides, and metal acetates.

15. The process of claim 14, wherein the catalyst is Pd/C.

16. The process of claim 11, wherein the hydrogen gas is provided in an amount that affords an intra-vessel gas pressure ranging from 1 bar to 20 bar.

17. The process of claim 11, wherein the solvent is selected from the group consisting of methanol, ethanol, propanol, isopropanol, butanol, tert-butanol, THF, 2-Me-THF, toluene, and ethyl acetate.

18. The process of claim 11, further comprising heating the catalyst to a temperature ranging from 25° C. to 100° C.

19. The process of claim 11, wherein no heat is supplied to any component of the continuous flow reactor.

20. The process of claim 11, further comprising purging the continuous flow reactor with an inert gas prior to addition of reactants and/or catalyst.

21. A bifurcated continuous flow process for producing hexahydrocannabinol from a cannabidiol starting material, the process comprising:
mixing a cannabidiol starting material with a solvent to form a solution;
adding an acid to the solution to form a reaction solution;
feeding the reaction solution through a first continuous flow loop;
passing the reaction solution through an energizing component to produce tetrahydrocannabinol;
feeding the reaction solution comprising tetrahydrocannabinol through a second continuous flow loop;
feeding hydrogen gas into the second continuous flow loop to form a combined feed comprising the hydrogen gas and the reaction solution comprising tetrahydrocannabinol; and
passing the combined feed over a catalyst bed within the second continuous flow loop to produce hexahydrocannabinol.

22. The process of claim 21, further comprising continuously flowing the reaction solution through the first continuous flow loop until a tetrahydrocannabinol yield of at least 95% has been reached.

23. The process of claim 21, further comprising continuously flowing the reaction solution through the first continuous flow loop until a Hexahydrocannabinol yield of at least 95% has been reached.

24. The process of claim 21, further comprising passing the reaction solution comprising tetrahydrocannabinol through a filter prior to feeding the reaction solution into the second continuous flow loop to remove at least a portion of the acid.
